(19) United States
(12) Patent Application Publication
(10) Pub. No.: US 2011/0104715 A1
(43) Pub. Date: May 5, 2011

(54) CYTOTOXIC PEPTIDES AND PEPTIDOMIMETICS BASED THEREON, AND METHODS FOR USE THEREOF

(76) Inventors: Dale E. Bredesen, Novato, CA (US); Shahrooz Rabizadeh, Los Angeles, CA (US)

(21) Appl. No.: 12/551,400

(22) Filed: Aug. 31, 2009

Related U.S. Application Data
(62) Division of application No. 10/472,812, filed on May 6, 2004, now abandoned.
(60) Provisional application No. 60/280,515, filed on Mar. 29, 2001.
(60) Provisional application No. 60/281,050, filed on Apr. 2, 2001.

(51) Int. Cl. G01N 33/68

(52) U.S. Cl. 435/7.21; 436/501

(57) ABSTRACT

In accordance with the present invention, it has been discovered that the β-amyloid precursor protein (APP), and two APP-like proteins (APLP1 and APLP2) are proteolytically cleaved by caspases in the C terminus to generate an approximately 31 amino acid peptide. It has been further discovered that the resultant C-terminal peptide is a potent inducer of apoptosis. Both caspase-cleaved APP and activated caspase-9 is present in brains of Alzheimer's disease patients but not in control brains. These findings indicate that caspase cleavage of APP and APP-like proteins leads to the generation of apoptotic peptides, which may contribute to the neuronal death associated with Alzheimer's disease. Accordingly, there are provided compositions and methods for modulating apoptosis.

### RELATED APPLICATIONS

This application claims the benefit of U.S. Provisional Application No. 60/280,615, filed Mar. 30, 2001, and U.S. Provisional Application No. 60/281,050, filed Apr. 2, 2000, the contents of both of which are hereby incorporated by reference herein in their entirety.

### FIELD OF THE INVENTION

The present invention relates to cytotoxic peptides, and the use thereof for developing agents which block undesired apoptosis, and the like. In particular, the present invention relates to methods for using peptides and peptidomimetics based thereon to induce apoptosis, or to prevent and/or inhibit undesired apoptosis. In yet another aspect, the present invention relates to methods for identifying and/or developing agents which induce and/or inhibit apoptosis.

### BACKGROUND OF THE INVENTION

Cell death in the central nervous system (CNS) occurs extensively in development, during normal aging and in some pathological states associated with degeneration of specific subsets of neurons. The majority of cell deaths in the developing nervous system occur by the activation of programmed cell death, and neural death in at least some disease states may involve components of the apoptotic pathway (Bredesen, Ann. Neurol. 38:839-851 (1995); Sperandio et al., Proc. Natl. Acad. Sci. USA 97:14376-14381 (2000); Yuan and Yankner, Nature 407:802-809 (2000). Elucidating the molecular mechanisms that initiate and control pathological cell death in the CNS should help in the development of interventions that may prevent or ameliorate degenerative CNS diseases.

The loss of hippocampal neurons is one of the prominent features of Alzheimer's disease (AD). The pathological hallmark of AD is the formation of senile plaques and neurofibrillary tangles in brain which is accompanied by substantial neuronal and synaptic loss in the neocortex. β-Amyloid precursor protein (APP) is a ubiquitously expressed membrane-spanning glycoprotein that is cleaved during its normal metabolism to generate the amyloid-β protein (Aβ), a 40 to 42 amino acid peptide that is the main constituent of senile plaques. The deposition of Aβ may account for the enhanced susceptibility of hippocampal and cortical neurons to premature death, since exposure of cultured human neuronal and non-neuronal cells to amyloidogenic Aβ peptide induces the activation of apoptotic cell death pathways (Cotman, Soc. for Neuroscience Satellite Symposium on Neural Apoptosis (1994); Cotman and Anderson, Mol. Neurobiol. 10:19-45 (1995); La Ferla et al., Nature Genet. 9:21-30 (1995)).

In addition to the cleavages that result in the formation of Aβ, APP can be cleaved at its C-terminus by caspases, a family of cysteine proteases central to the execution of apoptosis (Lyckman et al., J. Biol. Chem. 273:11100-11106 (1998); Gervais et al., Cell 97:395-406 (1999); LeBlanc et al., J. Biol. Chem. 274:23426-23436 (1999); Pellegrini et al., J. Biol. Chem. 274:21011-21016 (1999)). In addition, it is possible that this C-terminal caspase cleavage, generating a 31 amino acid fragment (C31), precedes and may favor the intramembrane cleavage that leads to the generation of Aβ.

The formation of Aβ and its subsequent deposition in senile plaques are viewed by many as the initiating events that lead to the cascade of pathological changes resulting in AD (Selkoe, Trends Cell Biol. 8:447-453 (1998)). Aβ is derived from APP by two or more proteolytic events mediated by β- and δ-secretase activities, and has been shown to be neurotoxic, with pro-apoptotic effects (Cotman, Neurobiol. Aging 18:S29-S32 (1998); La Ferla et al., supra; Yankner, Neuron 16:921-932 (1996)). However, whether Aβ cytotoxicity occurs in vivo has not been determined. Indeed, Aβ is not likely to be the only cause of synapse loss and neuronal loss in AD, and may not even prove to be the main cause; several other factors have been proposed as mediators of AD pathogenesis, including oxidative damage, inflammation, mitochondrial dysfunction and apolipoprotein E, among others. Not only is the cause of the neuronal and synaptic loss incompletely understood, but also the mode of cell death that occurs in AD is controversial. Apoptosis has been reported in the brains of patients with AD (Cotman (1998), supra), but this does not seem to be a general process. Thus, both the mechanisms and cellular pathways responsible for neuronal death in AD are still poorly defined.

Accordingly, there remains a need in the art for compositions and methods to control apoptosis, in particular for application in Alzheimer's disease. The present invention fulfills this need and further provides related advantages.

### SUMMARY OF THE INVENTION

In accordance with one aspect of the present invention, there are provided peptide compositions or peptidomimetics thereof, wherein the peptide is a potent inducer of apoptosis. In specific embodiments, the peptide is derived from β-amyloid precursor protein (APP), APP-like protein 1 (APLP1), or APP-like protein 2 (APLP2).

In accordance with another aspect of the present invention, there are provided methods for inducing apoptosis in a target cell using an effective amount of a peptide or peptidomimetic thereof that is a potent inducer of apoptosis. In a preferred embodiment, the target cell is a neural cell, such as a neuron or glial cell.

In accordance with yet another aspect of the present invention, there are provided methods for reducing or inhibiting apoptosis of cells containing β-amyloid precursor protein (APP) or an APP-like protein by blocking cleavage of the precursor that releases a C-terminal peptide fragment. In specific embodiments of such methods, apoptosis is reduced or inhibited in neural cells, such as neurons or glial cells. In preferred embodiments, cleavage is blocked by small molecule compounds such as peptides, antisense peptides, peptidomimetics, antibodies, antagonists, antisense nucleic acids, and the like.

In accordance with another aspect of the present invention, there are provided methods for reducing or inhibiting apoptosis of cells containing β-amyloid precursor protein (APP) or an APP-like protein by inactivating the C-terminal peptide fragment formed by cleavage of the precursor protein as it is formed. In specific embodiments of such methods, apoptosis is reduced or inhibited in neural cells, such as neurons or glial cells. In preferred embodiments, the peptide fragment is inactivated by degrading the peptide into inactive fragment(s) or by combining the peptide with a chelator, such as an antibody.

In accordance with still another aspect of the present invention, there are provided methods of treating a subject in need thereof, comprising administering a therapeutically effective amount of a molecule capable of blocking the cleavage of APP or an APP-like protein or capable of inactivating the C-terminal peptide fragment generated by cleavage of the precursor protein. In a preferred embodiment, the subject in need thereof has Alzheimer's disease.

In accordance with another aspect of the present invention, there are provided methods of identifying small molecules that will block cleavage of APP or an APP-like protein, comprising determining which small molecules will compete for specific binding to the APP or APP-like protein.

### BRIEF DESCRIPTION OF THE FIGURES

FIG. 1 collectively illustrates APP interaction with and cleavage by caspases in cultured cells. In FIG. 1a, APP (a type-1 integral membrane glycoprotein) is illustrated, as are fragments produced by caspase cleavage in the intracellular region of APP; and the antibodies used herein. Cleavage of APP at the caspase consensus site, VEVD/A, after the aspartic acid, is predicted to yield an N-terminal protein of 664 amino acids (APPΔC31) and a C-terminal peptide of 31 amino acids (C31), which contains the APP internalization signal NPTY (SEQ ID NO:5). 5A3 and 1G7 are monoclonal antibodies against the same extracellular region of APP (mixed together to detect the full-length APP and APPΔC31); CT15 is a polyclonal rabbit antibody against the C-terminal 15 amino acids of APP; 26D6 is a monoclonal antibody against Aβ₁₋₁₂; and α-1 is a polyclonal antibody against APP amino acids 649-664.

FIG. 1b shows that APP interacts with caspases. APP was co-immunoprecipitated with caspases-6, -7, -8 and -9 from 293T cells co-transfected with APP and the respective caspases tested. Catalytic mutant caspases with the active site cysteine mutated to alanine were used so that co-immunoprecipitation could be done without cell death induction. Monoclonal anti-FLAG M2 was used for the co-immunoprecipitation of FLAG-tagged caspases. Western blot analysis used monoclonal antibody (5A3/1G7) for APP. Lane 5 shows that caspase-8 does not interact with APPΔC31. Lane 7 shows cells transfected with APP and immunoprecipitated and probed with monoclonal antibody 5A3/1G7 as a positive control. Quantitative densitometry analysis showed that C8 had an intensity about 200% of that of the other caspases tested (C6, 1.2; C7, 1.0; C8, 2.2; C9, 1.1).

FIG. 1c shows that APP is cleaved in 293T cells co-expressing APP and caspase-8. Cell lysate samples were immunodepleted with CT15 (Immunodep CT), then immunoprecipitated with either the mixture of monoclonal mouse antibodies 5A3 and 1G7 (MAb) or CT15 (CT). After immunodepletion, a principal C-terminal truncated species is evident (lane 1); immunodepletion removes most of the full-length APP species (lane 2). The faint bands migrating at a higher molecular weight (lanes 1 and 3) represent endogenous APP₇₅₁ present in 293T cells.

FIG. 2 collectively illustrates the results of cell death and viability assays in cultured cells expressing various APP and C-terminal fragment (CTF) constructs. FIG. 2a shows cell death in N2a cells transfected with various constructs. Expression of C31 increases cell death compared with control (P<0.001 by one-way ANOVA (P<0.0001; F=44.838), post-hoc Tukey-Kramer). Transfection of cells with APP (P<0.001) or V642F (P<0.001) also causes significant cell death compared with control.

FIG. 2b shows cell death in 293T cells when various constructs co-expressed with caspase-7 or -8. Caspase-8 is significantly more toxic when co-expressed with APP in 293T cells than caspase-8 or APP expressed alone (P<0.001 by two-way ANOVA (P<0.00001; F=186.9), post-hoc Tukey HSD).

FIG. 2c shows the viability of 293T cells in which apoptosis was induced with tamoxifen in the presence of various constructs. C100 causes more cell death than APP (P<0.001, one-way ANOVA (P<0.0001; F=157.58), post-hoc Tukey-Kramer) but slightly less cell death than C31 (P<0.05). C100-D664A abolishes all of the cytotoxic effects of C100, compared with mock transfection with pcDNA3 (P>0.05).

FIG. 3 illustrates in vivo caspase-9 activation in AD and control brains. Crude synaptosomal preparations were immunoprecipitated with a polyclonal antibody against caspase-9, followed by western blot analysis with an activation-specific antibody against caspase-9. Lane 1 shows Hela cells transfected with caspase-9 and treated with the pan-caspase inhibitor zVAD.fmk. Lane 2 shows caspase-9 transfected staurosporine-treated Hela cells. Lane 3 shows active recombinant caspase-9. In all five AD patients and one neurologically affected non-AD control patient (with normal-pressure hydrocephalus and dementia) there are activated caspase-9 p10 fragments(*).

FIG. 4 demonstrates APP C31 toxicity to both neurons and glial cells in primary hippocampal cultures. Cultures were treated with various concentrations of the penetration peptide conjugated to C31 (DP-APPC31) or 10 μM of DP, immunostained 24 hours later with antibodies specific for the neuronal marker NeuN or the glial marker GFAP. Relative area values for NeuN and GFAP immunoreactivity were obtained by image analysis using the Simple PCI software (Compix, Inc., Philadelphia). The broad spectrum caspase inhibitor BAF blocked the toxicity of the DP-APPC31 conjugate at 10 μM.

FIG. 5 demonstrates that transduction of APP C31 induces overall cell death in hippocampal cultures. Primary hippocampal cultures were transduced with the penetratin peptide (delivery peptide DP) or the DP-APPC31 conjugate at various concentrations and then assayed for viability 36 hours later by the MTT assay.

FIG. 6 collectively shows APP C31 induced cell death in primary hippocampal culture, FIG. 6a measures viability of the cultures by the trypan blue exclusion method thirty hours after transduction with the peptides. FIG. 6b measures condensed, fragmented nuclei by staining cultures with 0.1 mg/ml Hoechst 33342 30 hours after transduction with the peptides.

FIG. 7 collectively shows that the C-terminal cleavage product of the APP homolog, APLP1, induces death in primary hippocampal cultures, in primarily neurons and not glial cells. FIG. 7a shows cultures treated with increasing concentrations of DP-APLP1C31 peptide in the presence or in the absence of the caspase inhibitor, BAF. Twenty-four hours later, the cultures were immunostained with antibodies specific for NeuN (light) and GFAP (dark). FIG. 7b shows primary hippocampal cultures transduced with the indicated concentrations of DP-APLP1C31 or DP alone. Thirty hours later, the viability of the cultures was determined by the trypan blue exclusion method.

### DETAILED DESCRIPTION OF THE INVENTION

In accordance with the present invention, it has been discovered that, in addition to Aβ, APP gives rise to a second cytotoxic, proteolytically derived fragment unrelated to Aβ. Furthermore, the toxicity of the APP C-terminal fragment, called C100, is attributable to this pro-apoptotic APP fragment. The mechanism of toxicity appears to be similar to that used by a class of cell death receptors called dependence receptors (Bredesen et al., Cell Death Diff. 5:365-371 (1998)); this class includes the common neurotrophin receptor p75^{NTR}, the netrin-1 receptor DCC (deleted in colorectal cancer), and the androgen receptor (Bredesen et al., supra; Mehlen et al., Nature 395:801-804 (1998); Rabizadeh et al., Science 261:345-348 (1993)). This second cytotoxic APP fragment is derived by caspase cleavage of APP at Asp664, mainly by caspase-8 and caspase-9, to generate a C-terminal peptide, called C31, comprising the C-terminal 31 amino acids of APP. C31 is potently pro-apoptotic by a mechanism that involves caspase amplification similar to that induced by DCC (Mehlen et al., supra). The presence of both caspase-cleaved APP fragments and activated caspase-9 species in brains of AD patients indicates that this process occurs in vivo. Thus, this cell death pathway mediated by C31 is also involved in physiological cell death.

Also provided herein is evidence that the 31 amino acid peptide released by caspase cleavage of the APP C-terminus is toxic in neuronal primary culture. These data support the notion that the release of the C31 peptide causes neuronal death in AD and plays a pathogenic role in the neurotoxicity associated with AD.

In accordance with another aspect of the invention, it has also been found that the two APP homologs, APLP1 and APLP2, can also be cleaved by caspases in vitro (the caspase recognition sequences at their C-termini are 100% conserved) and a synthetic peptide of APLP1-C31 delivered into primary cultures is preferentially toxic to neurons as compared to glial cells. The LD₅₀ for neurons is around 3 micromolar, compared with an LD₅₀ of 35-40 micromolar for glial cells.

Accordingly, the present invention provides peptides having the sequence of the C-terminal peptide of cleaved APP (AAVTPEERHLSKMQQNGYENPTYKFFEQM QN; SEQ ID NO:1) or a peptide having at least 80% sequence identity therewith; the sequence of the C-terminal peptide of cleaved APLP1 (PMLTLEEQQLRELQRHGYENP TYRFLEERP; SEQ ID NO:2) or a peptide having at least 80% sequence identity therewith; the sequence of the C-terminal peptide of cleaved APLP2 (PMLTPEERHLNK MQNHGYENPTYKYLEQMQI; SEQ ID NO:3) or a peptide having at least 80% sequence identity therewith, or a peptidomimetic of any of the above peptides, wherein said peptide or peptidomimetic is a potent inducer of apoptosis. Preferably an invention peptide has at least 90% sequence identity with SEQ ID NO:1, SEQ ID NO:2 or SEQ ID NO:3. An invention peptide may also have an amino acid sequence that differs from SEQ ID NO:1, SEQ ID NO:2 or SEQ ID NO:3 by conservative substitutions of one or more residues thereof.

The term “peptidomimetic” as used herein refers to a non-peptide small molecule that exhibits structural similarity to a peptide and has peptide-like properties. Examples include traditional peptides that contain non-amino acid moieties, or alternative linkages. The term “identity” refers to the exact same sequence of amino acids, while the term “similarity” allows for conservative amino acid substitutions, for example, a non-polar amino acid substituted for another non-polar amino acid, or a charged for a charged, etc.

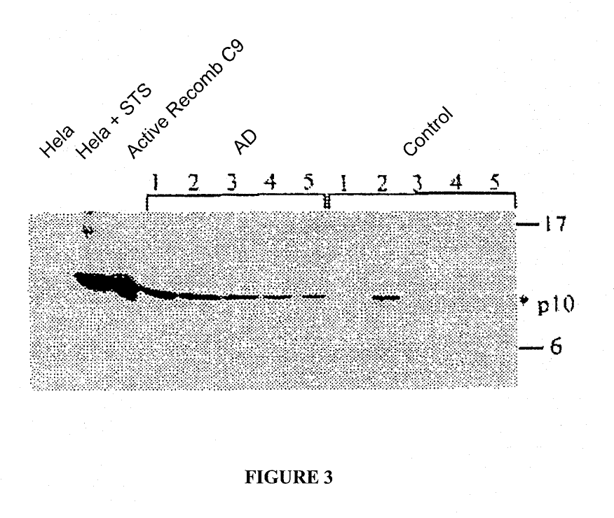

The caspase-generated fragment that comprises the C-terminal 31 amino acids of APP (C31) is a cytotoxic peptide that sensitizes cells to other stressful stimuli in a concentration-dependent manner. The present invention also provides evidence for cleavage of APP at the intracellular caspase site, D664, in the brains of patients with AD but not control patients (see FIG. 3). Taken together, these data strongly suggest that the cleavage of the C-terminal portion of APP plays an important role in the neural toxicity observed in AD pathogenesis, both by increasing the production of the toxic Aβ peptide and by generating a pro-apoptotic C-terminal fragment. Activation of caspases as a result of stress such as that induced by the accumulation of Aβ at neuronal terminals is, therefore, seen to provide the trigger for a ‘spiral’ of toxicity in which APP is cleaved at its C-terminus and generates an additional toxic fragment. Consistent with this mechanism, mice expressing an APP transgene carrying two point mutations linked to autosomal forms of familial AD develop neurological symptoms and extensive neuronal death in the absence of significant Aβ accumulation or amyloid plaque formation (Mucke et al., J. Neurosci. 20:4050-4058 (2000)).

Accordingly, the present invention provides methods of reducing/inhibiting apoptosis of a cell containing β-amyloid protein precursor (APP) or an APP-like protein, said method comprising blocking cleavage that releases a C-terminal peptide fragment. In certain embodiments, cleavage is blocked by small molecule compounds such as peptides, antisense peptides, peptidomimetics, antibodies, antagonists, antisense nucleic acids, and the like. In preferred embodiments the cell is a neural cell, such as a neuron or a glial cell.

In an alternative embodiment, the invention provides methods of reducing/inhibiting apoptosis of a cell containing β-amyloid protein precursor (APP) or an APP-like protein, said method comprising inactivating the C-terminal peptide fragment as it is formed. The peptide fragment is inactivated by degrading the peptide into inactive fragment(s) thereof, or by combining the peptide fragment with a chelator therefor, such as an antibody. In preferred embodiments the cell is a neural cell, such as a neuron or a glial cell.

APLP1 and APLP2 are members of the APP family of proteins, collectively “APP-like proteins”. However, the sites required for γ and β-secretase cleavage of APP are not conserved in either APLP1 or APLP2. These molecules therefore do not have the capacity to generate β-amyloid-like peptides. However, the C-terminal caspase cleavage site that allows for the generation of APP C31 is conserved in both APLP1 and APLP2. For APLP1, the P4-P1′ positions would be VEVDP, and for APLP2, the P4-P1′ positions would be VEVDP while in APP, the P4-P1′ positions are VEVDA. These sequences, like those in APP, fit well with previously described caspase cleavage sites for the initiator/apical caspases such as caspase-8 and caspase-9. The predicted APLP1-C31 peptide is 52% identical and 77% similar and the predicted APLP2-C31 is 71% identical and 83% similar to the APP C31 peptide.

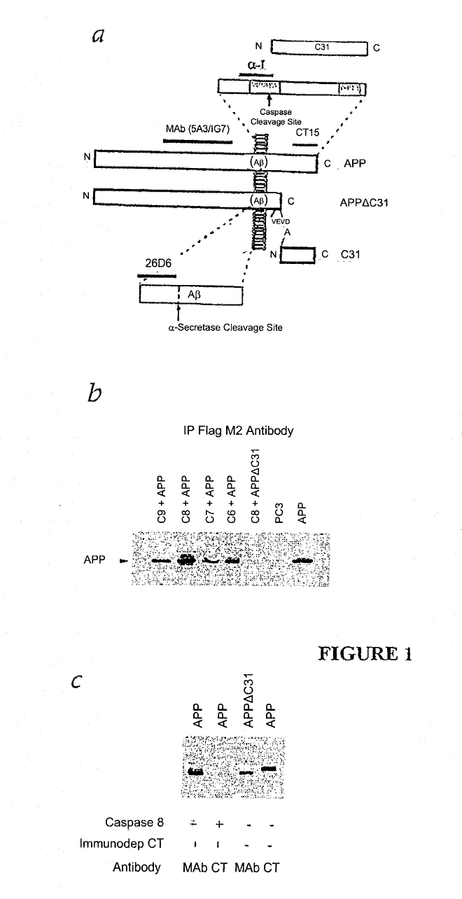

Using an in vitro cleavage assay, it has been found that caspases-3, -6 and -8 are capable of cleaving APP, and the cleavage by caspase-3 is blocked by mutation of Asp664 to Glu, confirming reports of caspase cleavage at this site (Barnes et al., J. Nenrosci. 18:5869-5880 (1996); Weidemann et al., J. Biol. Client. 274:5823-5829 (1999); Gervais et al., supra; Pellegrini et al., supra; LeBlanc et al., supra). After co-transfection of APP and caspases-6, -7, -8 or -9, complexes of APP and caspases formed, as shown by co-immunoprecipitation (see FIG. 1b). Moreover, in cultured cells, APP was cleaved by both caspase-8 and caspase-9. For full-length APP, caspase cleavage would lead to two fragments: an N-terminal fragment of 664 amino acids (APPΔC31) and a C-terminal fragment (CTF) of 31 amino acids (C31). In 293T cells co-expressing APP and the respective caspase zymogens (that is ‘pro-caspase’, the relatively less-active caspase precursors), a C-terminal-deleted APP fragment consistent with APPΔC31 (see FIG. 1c) was detected. Expression of a mutant APP, D664A, in which the probable caspase site was mutated to Ala, inhibited the ability of caspase-8 to cleave APP. Identical results were obtained when caspase-9 was co-expressed with APP. These results indicate that both caspase-8 and caspase-9 are capable of cleaving APP between residues 664 and 665. In contrast, caspases-3, -6 or -7 did not result in cleavage when tested in the same manner.

In accordance with another aspect of the present invention, there are provided methods for inducing apoptosis in a target cell, said methods comprising contacting target cell with an effective amount of a peptide having the sequence of the C-terminal peptide of cleaved APP or a peptide having at least 80% sequence identity therewith, the sequence of the C-terminal peptide of cleaved APLP1 or a peptide having at least 80% sequence identity therewith, the sequence of the C-terminal peptide of cleaved APLP2 or a peptide having at least 80% sequence identity therewith, or a peptidomimetic of any of the above peptides. In preferred embodiments the target cell is a neural cell, such as a neuron or glial cell. An “effective amount” as used herein refers to that amount of a peptide which is capable of causing cell death by apoptosis by any standard test as is known in the art, for example, the MTT assay as provided in the examples below.

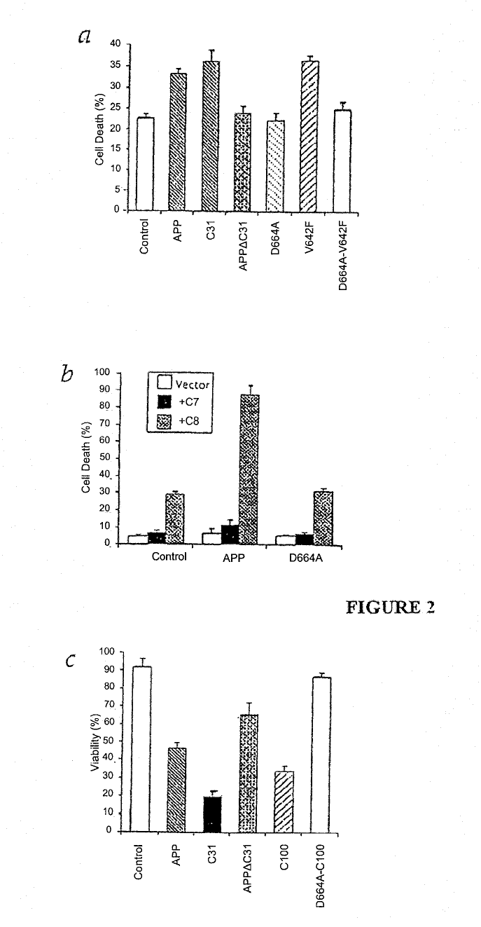

The effects of caspase-mediated cleavage of APP on cell death were evaluated by expressing wild-type APP₆₉₅, D664A, V642F, APPΔC31 and C31 (the predicted C-terminal APP fragment released after caspase cleavage) in 293T and N2a cells. Expression of wild-type APP and V642F had a pro-apoptotic effect (see FIGS. 2a and 2b) after staurosporine or tamoxifen induction, although the V642F mutant did not show a significantly greater pro-apoptotic effect than wild-type APP (see FIG. 2a). Further analysis showed that expression of C31 but not APPΔC31 produced the pro-apoptotic effects after stimulation by staurosporine that may even exceed those obtained with either APP₆₉₅ or the APP V642F mutation (see FIGS. 2a and 2c). Expression of the D664A mutation similarly led to inhibition of the proapoptotic effects (see FIGS. 2a and 2c).

The presence of APP potentiated apoptosis initiated by caspases. In 293T cells transfected with caspase-8 zymogen, cell death was significantly greater than in basal conditions or cells transfected with caspase-7 (see FIG. 2b). However, co-expression of caspase-8 zymogen and APP considerably increased cell death relative to the conditions described above, indicating a synergistic effect of APP and caspase-8 on cell death (see FIG. 2b). This effect was completely dependent on cleavage of APP at Asp664, and thus presumably C31, as the APP mutant D664A failed to show the additive effects on cell death (see FIG. 2b). Thus, the generation of C31 seemed to amplify the cell death program initiated by caspase-8.

Finally, expression of C31 also induced apoptosis in basal conditions without further cellular insults. In N2a neuroblastoma cells, expression of C31 alone, without tamoxifen or staurosporine, was strongly correlated with annexin V (Chan et al., J. Neurosci. Res. 57:315-323 (1999) staining (76±10% of C31-positive cells were annexin V-positive). This immunoreactivity was indicative of apoptosis, as the cells were impermeant to propidium iodide. However, cells transfected with APP or with pcDNA3 (mock transfection) were generally negative for annexin V conjugated to fluorescein isothiocyanate (annexin V-FITC) (13±5% were positive for both APP and annexin V)(P<0.001, two-tailed t-test). Treatment with zVAD.fmk decreased annexin V staining of C31 transfected cells to control levels. Therefore, these results show that release of a C-terminal caspase-cleaved APP fragment, presumably C31, consistently resulted in a pro-apoptotic phenotype in cultured cells.

C31 can theoretically be generated either from full-length APP or APP CTFs, the latter derived from α-, β- or δ-secretase cleavages of APP. β-secretase-cleaved APP, the so-called C100 (or C99) CTF, is cytotoxic (Oster-Granite et al., J. Neurosci. 16:6732-6741 (1996); Yankner et al., Science 245:417-420 (1980); Fukuchi et al., Neurosci. Lett. 154:145-148 (1992); Sopher et al, Mol. Brain. Res. 26:207-217 (1994)) and is increased in neurons expressing disease-associated APP mutations (Oster-Granite et al., supra). Here, C100, like APP, was cleaved by caspase-8 and caspase-9. In addition, the effects of wild-type and mutant C100 constructs (D664A-C100) on cell death were analyzed (see FIG. 2c). As expected, C100 had a pro-apoptotic effect on N2a cells (see FIG. 2c). However, the C100 caspase mutant D664A-C100 produced no cytotoxicity, reducing cell death to the levels obtained with control transfection. These observations therefore indicate that the reported cytotoxic properties of C100 may be entirely due to the generation and release of C31 and its subsequent amplification effect on the cell death program.

Having established that APP can be cleaved by caspase-8 and caspase-9 in cultured cells and that cell death is potentiated by this cleavage event, it was next sought to determine whether this process occurs in the brains of patients with AD. First, the pattern of these two caspases was examined by immunochemistry in mouse brain tissue. Adult mouse brain tissue was immunostained with antibodies raised against the uncleaved forms of caspase-8 and caspase-9. Immunoreactivity for these caspases was readily detectable in almost all neurons in brain, including neocortex, hippocampus and diencephalon. Although the staining was abundant in the perikarya for both caspases, proximal as well as distal neuronal processes were prominently stained by the antibody against caspase-9, indicating transport of caspase-9 to distal sites.

The studies described above showed that both APP and its CTFs derived from α- or β-secretase are substrates for caspases. Therefore, polypeptides that result from cleavage of APP CTFs (CTFΔC31) rather than from full-length APP were focused on, because these small, caspase-generated fragments are resolved much better by SDS-PAGE. As expected, CTFΔC31 fragments derived from caspase cleavage in cultured cells were detected. However, using whole-brain homogenates from mid-frontal cortex of both AD and control brain tissue, caspase-derived APP fragments were not detected. Although APP is mainly located in the intermediate compartments in cultured neurons (Caporaso et al., J. Neurosci. 14:3122-3138 (1994)), it is nonetheless also enriched from synaptosome preparations (Marquez-Sterling et al., J. Neurosci. 17:140-151 (1997)). Because caspase-9 is apparently distributed into neuronal processes as well, isolation of synaptosomes may enrich for caspase fragments. Indeed, in crude synaptosome samples obtained from mid-frontal regions of AD brains, multiple APP fragments were detected. The immunologic profile was such that it was possible to detect a fragment consistent with caspase cleavage of an α-secretase-derived CTF in AD brain tissue. Specifically, this fragment was recognized by the antibody against intracytoplasmic (I) APP but not by the antibody against the APP C terminus (CT15), indicating the absence of the C terminus. Moreover, this fragment was present in five of five AD brains examined but was absent in all control brains. Finally, this fragment was also detected in the brain of one adult with Down syndrome.

In addition to showing the presence of caspase cleavage of APP in brain tissue, the present invention provides evidence of caspase activation in the same tissue. The generation of C31 and its proposed downstream cytotoxic effects should be related to caspase activation, otherwise APP would not be cleaved. Effector caspase-3 and effector caspase-6 were not able to cleave APP and are therefore unlikely to initiate the proposed C31-mediated cell death. Thus, focus was placed on evidence of caspase-8 or caspase-9 activation in brains of AD patients.

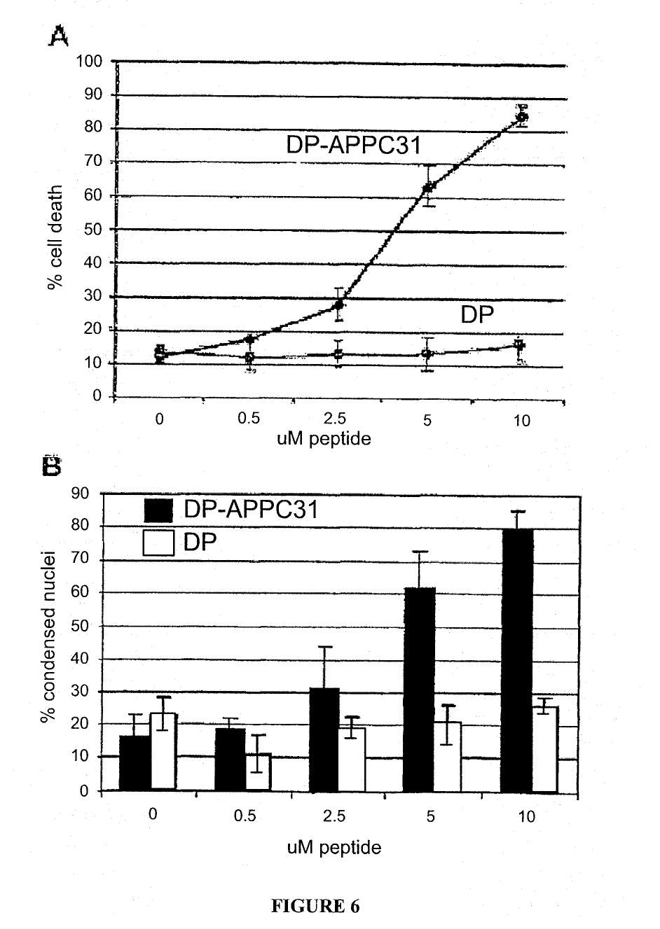

In the crude synaptosomal preparations of AD and control brain tissues, it was not possible to detect caspase-8 by immunoblotting. This was consistent with the predominant perikaryal and sparse neuritic staining of caspase-8 in mouse brains, and therefore no further examination for caspase-8 was carried out. Caspase-9 in its full-length zymogen form was present in crude synaptosome samples, as shown by western blot analysis. Therefore, specific investigation for activated p10 fragments of caspase-9 (that is the small subunit resulting from cleavage and activation of caspase-9) was then carried out using an activation-specific antibody against caspase-9, referred to as 315/316. Indeed, there was a fragment about 10 kDa in size, co-migrating with a band from staurosporine-treated Hela cells transfected with caspase-9 and a caspase-9 p10 recombinant fragment, in the AD brain samples (see FIG. 6). Moreover, this activated caspase-9 fragment was present in five of five AD brains examined. However, this activated caspase-9 fragment was not found in four of the five control brains (see FIG. 6, control, 1 and 3-5). The one brain with positive results was from a neurologic control subject with dementia (normal-pressure hydrocephalus) but without AD changes (see FIG. 6, control, 2).

A central feature of AD pathology is the profound loss of neurons in cortex, although the mechanisms responsible for neuronal death are unclear. Given recent studies of pro-apoptotic receptors (Bredesen et al., supra; Mehlen et al., supra; Ellerby et al., J. Neurochem. 72:185-195 (1999); Rabizadeh et al., Science 261:345-348 (1993)), it was next determined whether APP is involved in physiological cell death by using a similar proteolysis-dependent mechanism. According to the present invention, it is shown that APP is a caspase substrate; caspase cleavage of APP at Asp664 generates a cytotoxic C-terminal APP fragment; the toxicity of C100 is dependent on caspase cleavage; in cultured cells, caspase-8 and caspase-9 were capable of cleaving APP; and both intracytoplasmic cleavage of APP (presumably caspase-mediated) and activation of caspase-9 occurs in the brains of AD individuals.

Consistent with recent reports (Barnes et al., supra; Weidemann et al., supra; Gervais et al., supra; Pellegrini et al., supra; LeBlanc et al., supra) APP was cleaved by caspases at Asp664 both in vitro and in cultured cells. Furthermore, catalytic mutants of caspases-6, -7, -8 and -9 co-immunoprecipitated with APP. However, only caspase-8 and caspase-9, but not caspases-3, -6 or -7, cleaved APP when co-expressed in cultured cells. Thus, interactions of various caspases with APP did not necessarily lead to APP proteolysis. This cleavage event produced two predicted fragments: an N-terminal fragment of 664 amino acids and a CTF of 31 amino acids. Consistent with this, an APP C-terminal-deleted fragment (APPΔC31) was present in cells co-expressing caspase-8 or caspase-9. Both APP and the α- and β-secretase cleaved CTFs were also substrates for caspase cleavage.

To determine the biological consequences of this cleavage, cell death assays were carried out in cultured cells. Expression of C31 was substantially more pro-apoptotic (in most cell death assays) than expression of either APP or V642F in the 293T and N2a cell lines. The small differences between experiments are probably due to the assay methods. Mutation of the caspase cleavage site abolished most of the pro-apoptotic effects of APP₆₉₅ and APP with the V642F mutation (see FIG. 2a). Furthermore, similar results were obtained when C100, rather than APP, was the (initial) substrate for caspase cleavage. Therefore, caspases may cleave either full-length APP or a C-terminal APP fragment, in both cases generating the cytotoxic C31 peptide. Thus, these findings provide evidence that the cytotoxicity of APP and its fragments, at least in cultured cells of a neuronal (N2a) or non-neuronal (293T) phenotype, results mostly from the generation of C31 by caspases.

In support of this, APP substantially enhanced the cell death induced by expression of caspase-8, but the non-cleavable mutant, APP-D664A, showed no such enhancement. Thus, even though the expression of caspase-8 alone was pro-apoptotic, the ability to generate C31 amplified the effect of caspase-8 in inducing cell death. The effect was completely dependent on APP cleavage by caspases. As C31 is not likely to be a product of constitutive APP processing, the results presented herein indicate that C31 may function by amplifying caspase activation, and thus the cell death program. As a result, exposure to pro-apoptotic stressors such as Aβ would be more likely to lead to cell death.

The C31 cytotoxic APP fragment disclosed herein may account for the cytotoxicity of the C100 fragment. C100, rather than Aβ, was the first cytotoxic fragment to be identified from APP (Yankner et al., supra) Expression of C100 in cultured cells and in transgenic mice results in significant neuronal death (Oster-Granite et al., supra; Yankner et al., supra). In addition, levels of C100 are also increased in neurons expressing various APP mutations (McPhie et al., J. Biol. Chem. 272:24743-24746 (1997)). The mechanism of C100 cytotoxicity has been a matter of debate, but the data presented herein indicate that it is mediated through caspase cleavage of the C-terminal APP fragment and the generation of C31 (or, conceivably, by non-caspase proteolytic cleavage of APP to generate a fragment similar to C31). Thus, in addition to generating increased levels of Aβ₁₋₄₂, APP mutations, through increased levels of C100, may provide more substrate for caspase cleavage, thereby enhancing C31 production and apoptosis induction. By this proposed mechanism, this last step may be normally relatively quiescent, but leads to a shift in the cellular ‘apostat’ (the likelihood that a cell will undergo apoptosis (Salvesen and Dixit, Cell 91:443-446 (1997)) such that any cytotoxic challenge would be more likely to result in cell death through C31-mediated amplification of caspase. The mechanism proposed herein does not exclude Aβ toxicity, and in fact complements proposed mechanisms that include Aβ toxicity. Indeed, C31 may function in concert with Aβ to produce the neuronal loss that characterizes AD.

Accordingly, the invention also provides methods of treating a subject in need thereof, said methods comprising administering a therapeutically effective amount of a molecule capable of blocking the cleavage of APP or an APP-like protein, or inactivating the C-terminal peptide fragment generated by cleavage of the precursor. In preferred embodiments, said subject has Alzheimer's disease.

Evidence of intracytoplasmic APP cleavage in vivo was first reported by detection of the APPΔC31 fragment in a single AD brain by immunostaining with an end-specific antibody (Gervais et al., supra). Biochemical evidence is provided herein of caspase cleavage of APP in five of five AD samples but not in any of the control samples. The results presented herein further show that the presence of caspase-cleaved APP fragments coincided with caspase-9 activation in AD brains but not in the four neurologically unaffected control brains. One neurologically affected, non-AD control brain that was also positive for caspase-9 activation but not APP cleavage was from an individual with normal-pressure hydrocephalus and dementia. Nonetheless, the presence of activated caspase-9 along with APPΔC31 fragments from the same synaptosomal preparations in all the AD brains examined provides compelling evidence that this caspase-mediated cleavage of APP occurs during the course of AD. The fact that the APP cleavage was detected in crude synaptosome preparations but not in whole-brain homogenates could simply be explained by the paucity of these fragments such that the synaptosomes represented a convenient way to enrich for APP. Alternatively, the finding may indicate that caspase activation and subsequent cleavage of APP occurs mainly in neurites. The latter interpretation is attractive because it would be consistent with the neuritic and synaptic abnormalities seen in AD brains (Masliah, J. Neural Trans. 53:147-158 (1998); Yang et al., Am. J. Pathol. 152:379-389 (1998); Mattson et al., Brain Res. 807:167-176 (1998)). It may also indicate that C31-mediated toxicity is one factor that contributes to synaptic degeneration.

Evidence of caspase activation in AD remains sparse and conflicting; so far, only activation of effector caspases (3 and 6) has been described (Chan et al., supra; Stadelmann et al., Am. J. Pathol. 155:1459-1466 (1999); Selznick et al., J. Neuropathol. Exp. Neurol. 58:1020-1026 (1999); LeBlanc et al., supra). Caspase-6 was shown to be activated in a single AD brain that was examined but not from a single control brain (LeBlanc et al., supra). Whether this result will extend to additional AD brains after further analysis is unclear. The data presented herein on the presence of activated caspase-9, an initiator caspase, may be particularly relevant. Although caspase-8 and caspase-9 are able to activate effector caspases (Salvesen and Dixit, supra; Thornberry and Lazebnik, Science 281:1312-1316 (1998); Stennicke et al., J. Biol. Chem. 273:27084-27090 (1998)), it seems that the activation of caspase-9 is not necessarily followed by downstream caspase activation in AD. Thus, it may be that the restricted nature of caspase-9 activation (that is, in presynaptic endings) does not lead to widespread caspase activation in perikarya. Alternatively, there may be other cellular mechanisms that limit the generalized activation of the caspase cascade. The latter concept would be consistent with evidence that there may be compensatory mechanisms in neurons that respond to the various chronic and perhaps accumulating insults that occur during neurodegenerative disorders (Cotman (1998), supra). Thus, neuronal death in neurodegeneration may represent a form of cell death that is neither classically necrosis nor apoptosis.

According to the present invention, it is shown that the APP fragment that is generated by caspase cleavage of the APP C-terminus at Asp664 is toxic to hippocampal and cortical neurons in primary culture. This peptide, C31, is relatively selectively toxic for the neuronal population, with a LC₅₀ of 1-2 μM for hippocampal neurons, 10-25 μM for astrocytes, and 50-100 μM for 293T human embryonic kidney cells. Moreover, primary cultures that have been exposed to otherwise sublethal concentrations of fibrilar Aβ demonstrate enhanced sensitivity to the C31 peptide, decreasing the LC₅₀ to <500 nM.

The presence of the C31 peptide in primary neuronal cultures triggers the activation of programmed cell death, as demonstrated by the condensation and fragmentation of nuclei in transduced cells and by the ability of the general caspase inhibitor BAF to delay the death process. This finding is compatible with the earlier finding that caspase-8 and caspase-9, but not caspase-3, were required for C31-induced cell death. The biochemical pathway(s) leading from C31 to caspases-8 and -9 and apoptosis activation is not yet known. However, it is compatible with the previous finding of caspase-9 activation in synaptosomal preparations from the brains of patients with AD, but not from control patients.

The evidence, taken as a whole, suggests that APP is cleaved both in cultured cells and in vivo, releasing not only the Aβ peptides, but also APP-C31, a relatively selectively neurotoxic peptide the toxicity of which is enhanced by otherwise sublethal concentrations of Aβ peptide. Thus the C31 peptide is a good candidate to play a role in the death of neurons associated with AD. It should be added that recent work from the d'Adamio Laboratory has shown that the APP-C57 peptide, which results from γ-secretase cleavage, may also be cytotoxic (Passer et al., J. Alzheimer's Dis. 2:289-301 (2000)). However, it is not yet clear whether generation of C31 is required for C57 toxicity, as was previously demonstrated for C100. It is also not yet clear whether the toxicity of C57 is relatively selective for neurons.

In accordance with another aspect of the present invention, there are provided methods of identifying small molecules that will block cleavage of APP or an APP-like protein, said method comprising determining which small molecules will compete for specific binding to APP or an APP-like protein.

The C-terminal part of APP has been shown to play a critical role in both APP internalization and according to the present invention, in the induction of cell death. This C-terminal fragment of APP harbors a NPTY (SEQ ID NO:5) motif required for the endocytosis of APP and consequent Aβ formation. On the other hand, phosphotyrosine-binding (PTB) domains bind to the NPTY motifs and may play a role in protein endocytosis. For instance, the protein Fe65, containing two PTB domains, has been reported to mediate APP endocytosis. Other proteins harboring PTB domains have been described to bind the NPTY motif. That is the case of X11, a neuron-specific protein that has been shown to bind in vivo to APP and compete for APP binding with Fe65. The overlapping of APP regions involved in the binding of Fe65 and X11 suggest the existence of competitive mechanisms regulating the binding of the various ligands to this cytosolic domain and hence represent novel therapeutic targets.

Also, in accordance with the present invention, it has been discovered that the C31 peptide derived from APP binds to the PTB domain of Fe65. Binding assays can be used to confirm this observation with respect to the C31 peptide derived from APLP1.

Using X-ray crystallographic information regarding C31, the 3 dimensional conformation of this peptide and its binding site was determined. By screening available databases of small molecule compounds, compounds can be identified with the potential to mimic the action of C31, i.e., to induce apoptosis and also conversely to block the binding site for C31, thereby blocking its apoptotic activity. Also candidates can be found that bind directly to C31 and inhibit its activity of caspase amplification and thereby inhibit apoptosis in neuronal cells. Exemplary compounds identified by these screening methods include antibiotics and flavonoids.

Thus, in accordance with another aspect of the present invention, a rational approach can be used for the development of small molecules that will compete for the specific binding of Fe65/APP and X11/APP (employing, for example, Catalyst, software from Molecular Simulations Inc.). For this purpose, the available crystallographic structure of X11/APP complex can be used and the Fe65/APP interaction modeled based on the X11/APP complex. Applying this approach, 145 potential pharmacophores have been identified from 5 databases containing more than 600,000 compounds. Four of these have a statistically significant docking score and can be grouped in two chemically distinct groups, i.e., flavonoids and antibiotics. Additional potential compounds contemplated for use in the practice of the present invention include small molecules such as, for example, peptides, peptidomimetics, antisense peptides, antibodies, antagonists, antisense nucleic acids, and the like.

The invention will now be described in detail by reference to the following non-limiting examples.

### Example 1

### Plasmid Construction and Mutagenesis

Wild-type human APP₆₉₅ was subcloned into pcDNA3 (Invitrogen, Carlsbad, Calif.). The mutation of the aspartate residue at codon 664 to glutamate (D664E) or alanine (D664A) and the familial Alzheimer disease mutation of valine to phenylalanine at codon 642 (V642F, or V717F by APP 770 numbering) was accomplished using the QuikChange method (Stratagene, La Jolla, Calif.). Three constructs encoding different lengths of the APP C terminus were made: APP-C125, APP-C100 and APP-C31. In APP-C125 and APP-C31, the constructs were generated by PCR from APP₆₉₅ to encompass the last 125 and 31 amino-acid residues, respectively. An ATG start codon was introduced before and in-frame with residue 571 (APP-C125) or residue 665 (APP-C31). APP-C100 comprises the signal peptide sequence of APP fused to the C-terminal 99 amino-acid residues beginning at the aspartate residue of Aβ. Three C-terminal APP deletion constructs were produced by PCR from the respective full-length cognates: APPΔC31, APP-V642F-ΔC31 and APP-C100-ΔC31. All APP expression constructs were subcloned into pcDNA3 (Invitrogen, Carlsbad, Calif.) and verified by sequencing.

With the QuikChange Site-Directed Mutagenesis Kit (Stratagene, La Jolla, Calif.), the following catalytic mutant caspases, which disable the catalytic cysteine residue, were generated: caspase-6 (C163A), caspase-7 (C186A), caspase-S(C360A) and caspase-9 (C287A).

### Example 2

### Cell Culture and Antibodies

Human embryonic kidney 293T cells were grown and maintained in Dulbecco's modified Eagle's medium supplemented with 10% fetal bovine serum at 37° C. and 5% CO₂. 293T cells were transiently transfected with plasmids using the calcium phosphate method.

Mouse N2a neuroblastoma cells were grown at 37° C. and 5% CO₂ in 45% Dulbecco's modified Eagle's medium and 45% OptiMEM I (Life Technologies) supplemented with 10% fetal bovine serum and 2 mM glutamine. Plasmid constructs were introduced into the N2a cells with the LipofectAMINE plus transfection reagent (Life Technologies) according to the manufacturer's instructions.

APP antibodies included the following: CT15, a polyclonal rabbit antibody recognizing the C-terminal 15 amino acids of APP (Sisodia et al., J. Neurosci. 13:3136-3142 (1993)); a mixture of two monoclonal mouse antibodies, 5A3 and 1G7, which recognize non-overlapping epitopes in the extracellular region of APP (Koo and Squazzo, J. Biol. Chem. 269:17386-17389 (1994)); (the two monoclonal antibodies were used together to increase sensitivity); and a monoclonal antibody 26D6 recognizing the Aβ peptide sequence of amino acids 1-12 (provided by M. Kounnas and S. Wagner of Merck Research Labs, San Diego, Calif.); rabbit polyclonal antiserum, α-1 (provided by D. Selkoe, Brigham and Women's Hospital, Boston Mass.), raised against a synthetic peptide of APP amino acids 649-664. Monoclonal ANTI-FLAG M2 was obtained from Sigma.

Rabbit antiserum Bur49, raised against human caspase-9, was generated as described (Krajewski et al., Proc. Natl. Acad. Sci. USA 96:5752-5757 (1999)). Rabbit antiserum 1890, raised against human caspase-8, was produced using the same methods as for Bur49. The specificity and affinity of antibodies 1890 and Bur49 to caspase-8 and caspase-9, respectively, were confirmed as described (Krajewski et al., supra). For subsequent immunohistochemistry, Bur49 was used at a dilution of 1:25,000 and 1890, at a dilution of 1:15,000.

Polyclonal antibody against caspase-9, directed against the entire caspase-9 zymogen, was used for immunoprecipitation (Stennicke et al., supra; Wolf et al., Blood 94:1683-1692 (1999)). Rabbit antiserum 315/316 (Biosource, Camarillo, Calif.) was used for subsequent western blot analysis. This antibody is specific for the N terminus of the cleavage site 315/316 of human caspase-9 and consequently detects the p10 fragment of active caspase-9. For caspase-8 immunoblotting, the monoclonal antibody B9-2 (PharMingen, San Diego, Calif.), recognizing amino acids 335-469 of caspase-8 fragment, was used.

### Example 3

### Induction of Apoptosis and Assessment of Viability

After transfection, apoptosis was induced in 293T cells as described (Ellerby et al., supra). After incubation of 293T cells (plated in six-well plates at a density of 5×10⁵ cells per well) in the calcium-phosphate-DNA solution for 20-24 h, the apoptosis-inducing agent tamoxifen was added at a final concentration of 50 μM. After 3 h more of incubation, cells undergoing cell death were quantified by the trypan blue method (Ellerby et al., supra).

Apoptosis of N2a cells was assessed by Hoechst staining and the MTS (3-(4,5-dimethylthiazol-2-yl)-5-(3-carboxymethoxyphenyl)-2-(4-sulfophenyl)-2H-tetrazolium, inner salt) assay according to manufacturers' instructions (Promega, Madison, Wis.). MTS is a cell proliferation assay that measures the number of viable cells for mitochondria activity (dye reduction), and therefore it indirectly measures cell viability. Apoptosis was induced with tamoxifen using the protocol described above for 293T cells. Cell viability was calculated by normalizing the absorbance value of each respective well to the absorbance value of the control well without transfection and expressed as percent viability of control cells.

For Hoechst staining, N2a cells were treated with 0.5 μM staurosporine for three hours, followed by a 5-minute incubation with 5 μg/ml bis-benzimide (Hoechst 33258; Molecular Probes, Eugene, Oreg.) as described (Shindler et al., J. Neurosci. 17:3112-3119 (1997)). Apoptotic cells, defined by abnormal morphology under ultraviolet visualization, were assessed in photomicrographs of transfected cells. Cells were counted in four random fields from each well of cultured cells (about 300-500 cells), in triplicate for each condition. The results are expressed as a percentage of apoptotic nuclei divided by the total number of cells. In some experiments, the cells were co-transfected with a green fluorescent protein control vector to monitor transfection efficiency, which was typically approximately 70%. Similar results were obtained when the results are expressed as a percentage of total cells or total transfected cells determined by the use of green fluorescent protein.

### Example 4

### In Vitro Protein Synthesis and Caspase Cleavage

In vitro transcription and translation used the Promega Coupled kit (Promega, Madison, Wis.). The constructs pcDNA3-APPC125 (C-terminal 125 amino acids of APP) and pcDNA3-APPC125-D664E (D664E mutation in APP₆₉₅) were translated, and the protein products were used to assess caspase cleavage. Cleavage with caspases-3, -6, -7, -8, -9 and -10 was done and assessed as described (Ellerby et al., supra).

### Example 5

### Caspase Interaction Assay in Cultured Cells

Cells were co-transfected with catalytic mutant caspases-6, -7, -8 or -9 and APP or the deletion construct APPΔC31. Cell lysates of co-transfected 293T cells were prepared by incubation of cells for 30 min on ice, with occasional vortexing, in Nonidet-P40 lysis buffer (0.1% Nonidet-P40, 50 mM HEPES, pH 7.4, 250 mM NaCl and 5 mM EDTA). For immunoprecipitation, samples were incubated for 12 h with a monoclonal ANTI-FLAG M2-Agarose affinity gel (Sigma) or with the mixture of monoclonal antibodies 5A3 and 1G7) and Sepharose A beads to bind FLAG-tagged mutant caspases or APP, respectively. The beads were washed three times by centrifugation and resuspension in Nonidet-P40 lysis buffer and were resuspended in Laemmli sample buffer. The immunoprecipitated proteins were resolved by 10% SDS-PAGE and were transferred to PVDF membranes for western blot analysis with monoclonal ANTI-FLAG M2 (Sigma) or monoclonal antibody against APP to detect mutant caspase or APP, respectively. The immunoblots were developed with peroxidase-conjugated secondary antibody and enhanced chemiluminescence. Quantitative densitometry used NIH Image (Version 1.61).

### Example 6

### Caspase Cleavage of APP in Cultured Cells

293T cells were co-transfected with constructs encoding wild-type APP or APP D664A mutant, and caspase-3, -7,-B or -9 zymogens. In some experiments, 40 μM zVAD.fmk (benzoxycarbonyl-Val-Ala-Asp-CH2F) was added to the cells during cotransfection of APP and caspase-8 or caspase-9. At 24 h after transfection, the cells were lysed in 1% Nonidet-P40. CT15 or a mixture of two monoclonal mouse antibodies, 5A3 and 1G7, was added to the lysate along with protein A-Sepharose beads (Zymed, San Francisco, Calif.) or antibody against mouse IgG-agarose beads (American Qualex, San Clemente, Calif.), respectively, for overnight immunoprecipitation. The samples of protein-Ig-bead complexes were washed twice in lysis buffer, mixed 1:1 with 2× sample buffer, and boiled for 5 min. The protein samples were separated by 5% PAGE and transferred to PVDF membranes. Immunoblotting used the mixture of monoclonal mouse antibodies 5A3 and 1G7. Secondary antibody against mouse was used for chemiluminescence to detect bound primary antibody as described before (Koo and Squazzo, supra).

### Example 7

### Immunocytochemistry

N2a cells were plated on glass cover slips treated with polylysine and were transfected as described above. At 24 h after transfection, cells were directly stained with annexin V-FITC for apoptosis in tissue culture media, according to the manufacturer's instructions (annexin V-FITC Apoptosis Detection Kit; Calbiochem, La Jolla, Calif.). After being stained with annexin V-FITC, cells were fixed for 20 min in 4% paraformaldehyde in phosphate-buffered saline and permeabilized in 0.5% Triton X-100 in phosphate-buffered saline for 5 min. After being blocked in 5% BSA in phosphate-buffered saline, the cells were incubated for 1 h at room temperature with CT15, diluted 1:2,000. In parallel, cells were stained with propidium iodide after annexin V staining and paraformaldehyde fixation but without permeabilization to verify membrane integrity; the combination of annexin V staining and membrane integrity indicated apoptosis without secondary necrosis. The primary antibody was detected with Texas Redo-conjugated secondary antibody against rabbit (Molecular Probes, Eugene, Oreg.). Negative controls, which included preimmune serum and cells transfected with pcDNA3 (mock transfection), were assayed in parallel using the protocol described above. The cells were analyzed by confocal microscopy using a BioRad MRC-1024 system. Cells in five random fields were counted for each slide, in duplicate for each condition. The number of cells positive and negative for APP or C31 and apoptosis (annexin V-FITC) were determined, and relative risk was calculated (RT=p1/p2)=concordant staining [(+/+) times (−/−)] divided by discordant staining [(+/−) times (−/+)].

Double-labeling of APP and annexin V in N2a cells after transfection was performed. N2a cells were transfected with either APP or C31 followed by staining with annexin V-FITC to visualize apoptotic cells. Transfected cells were immunolocalized with CT15 followed by Texas Red conjugated secondary antibody to visualize APP or C31 expression. When expressed in N2a cells, C31 but not APP is associated with apoptosis. A high proportion of C31 transfected cells are positive for annexin V-FITC (green outline of the cell; RR=6.3), whereas in APP-expressing cells, annexin V-FITC staining is sparse (RR=0.77). In control cells transfected with pcDNA3 (mock transfection; control), there is no staining with annexin V-FITC and essentially no staining of endogenous APP at this antibody dilution. Images are representations of four different fields of view of each of three separate experiments.

For immunostaining of caspase-8 and caspase-9, free-floating tissue sections of mouse cerebral cortex and hippocampus were incubated with the appropriate polyclonal antibodies as described using a peroxidase system (Krajewski et al., supra). Brightfield images were obtained with Nikon Inverted E-300 Microscope.

Caspase-8 and caspase-9 are expressed in the frontal cortex and hippocampus of mice. Mouse frontal cortex and CA1 of hippocampus show neurons immunoreactive to antibodies against caspase-8 or caspase-9. The immunoreactivity is most abundant in the neuronal perikarya with antibodies against caspase-8 or caspase-9, whereas proximal neuronal processes are also immunostained by antibody against caspase-9. Control staining with pre-immune serum showed no background staining in the cerebral cortex.

### Example 8

### In Vivo Caspase Cleavage of APP in AD and Control Brains

For the in vivo study, brains of AD and control patients were obtained from the Alzheimer Disease Research Center at Johns Hopkins University. Diagnosis of AD was established by both CERAD (Consortium to Establish a Registry for Alzheimer's Disease) and NINDS/ADRDA (National Institute of Neurological Disorders and Stroke/Alzheimer's Disease and Related Disorders Association) criteria. The AD patients ranged in age from 72 to 86 years; the unaffected control subjects were 19, 40, 66 and 85 years old. The individual with normal-pressure hydrocephalus was 63 years old at death. Crude synaptosomes were prepared from the mid frontal cortex of the frozen tissue (Hui et al., J. Biol. Client. 273:31053-31060 (1998)). Approximately 2.5 g of gray matter was homogenized in 10% sucrose containing 1 mM dithiothreitol. The homogenate was centrifuged at 700 g for 20 min and the pellet was rehomogenized and centrifuged again at 700 g for 20 min. The two supernatants were combined and centrifuged at 10,000 g for 30 min to obtain the crude synaptosome pellet. The synaptosome pellet was subsequently lysed in the extraction buffer (50 mM HEPES, pH7.6, 250 nM NaCl, 0.1% Nonidet-P40, 50 mM EDTA and 0.5 mM dithiothreitol) with protease inhibitors. The lysate was precleared with protein A-Sephorose beads and incubated for 48 h with antibody against intracytoplamic (I) and protein A-Sepharose beads. The sepharose beads were collected and washed four to five times. Proteins immunoprecipitated were separated by 15% tricine gel electrophoresis and visualized by western blot analysis with CT15 or intracytoplasmic (I) APP.

To verify the identity of APP fragments generated by caspases, CTFs of APP were first assessed by western blot analysis for cleavage in cultured cells. In C100-transfected cells, there are two species of CTFs, one generated by β-secretase; the other, by α-secretase. As expected, both species are immunoreactive to CT15 and antibody against intracytoplasmic APP (α1), whereas only the fragment generated by β-secretase is positive for 26D6. In APP-transfected cells, the CTFs are mostly generated by α-secretase, as shown by positive immunoreactivity to CT15 and α1 but negative reactivity to 26D6. After co-transfection of C100 with caspase-8, CTFs of β- and α-secretase missing the epitopes of CT15 are generated. The immunoreactive profiles of 13-CTFΔC31 fragment and α-CTFΔC31 are as expected: the β-CTFΔC31 fragment is positive for 26D6 and α1, but negative for CT15; and the α-CTFΔC31 fragment is positive for only α1. Co-transfection of APP with caspase-8 generates mainly α-CTFΔC31 and some α-CTF.

Crude synaptosomal preparations were immunoprecipitated with α1 followed by western blot analysis for APP CTFΔC31 fragments. Immunoprecipitated products of 293T cell lysates co-transfected with C100 and caspase-8 were assayed next to the AD and control samples. In all five AD patients, α-CTFΔC31 is present and its identity is consistent with the immunologic profile of caspase-cleaved fragments: positive immunoreactivity for α-1 and negative immunoreactivity for CT15. In control tissues, this fragmentation is absent. Synaptosome samples from 10 subjects were assayed for synaptophysin immunoreactivity to verify equal loading of protein.

### Example 9

### In Vivo Detection of Caspase-9 Activation in AD and Control Brains

Crude synaptosomal preparations were analyzed for the presence of caspase-9 activation. The same crude synaptosome samples prepared from AD and control patients were subjected to immunoprecipitation (1:100 dilution) with the polyclonal antibody against caspase-9. The immunoprecipitates were separated by 15% Tris-glycine SDS-PAGE and immunoblotted with the activation-specific antibody against caspase-9, 315/316 (Biosource, Camarillo, Calif.). As a negative control, HeLa cells, transfected with caspase-9 zymogen, were treated with 40 μM zVAD.fmk (pan-caspase inhibitor). HeLa cells transfected with caspase-9 zymogen and treated with 1 μM staurosporine for 5 h served as a positive control for caspase-9 activation. For caspase-8 immunoblotting, the monoclonal antibody B9-2 (PharMingen, San Diego, Calif.), recognizing amino acids 335-469 of caspase-8 fragment, was used.

### Example 10

### Hippocampal Cultures

Hippocampal or cortical neurons derived from 17-day old rat embryos were plated in modified minimum essential media (MEM-PAK) supplemented with 5% horse serum. Three days later, the cultures were treated with 10 μM cytosine arabinoside (AraC). Twenty-four h later the cells were treated with the peptide conjugates and incubated for an additional 24 or 48 h. Cells were incubated in the presence of 50 μM of the general caspase inhibitor BOC-Asp(Ome)-FMK (BAF) for 30 min prior to addition of peptides or with an equivalent volume of dimethylsulfoxide (DMSO) and then maintained in the presence of the same concentration of the inhibitor for the duration of the experiment. In all experiments involving Aβ, a 1 mM Aβ42 stock solution was used that had been incubated at 37° C. for 48 h and then stored at 4° C. to allow for the formation of Aβ fibrils. Aβ42 was purchased from AnaSpec (San Jose, Calif.).

### Example 11

### Peptide Delivery into Cells

Peptides were synthesized and purified at the Stanford University Protein and Nucleic Acid (PAN) Facility. All peptide stocks were solubilized in water at 1 or 10 mM concentration. The delivery peptide derived from the Drosophila Antennapedia homeodomain (RQIKIWFQNRRMKWKK; SEQ ID NO:4) (Dorn et al., Proc. Natl. Acad. Sci. USA 96:12798-12803 (1999) also called penetratin (Nakagawa et al., Nature 403:98-103 (2000)), was cross-linked via an N-terminal Cys-Cys bond to the 31 amino acid peptide generated by caspase cleavage of APP (DP-APPC31) or APLP1(DP-APLP1C31), to an irrelevant peptide (C-N), or to itself (DP-DP) at the Stanford University PAN facility. Cargo peptides are released from the carrier by reduction of the disulfide bond in the intracellular environment. No toxicity has been observed in transduction experiments using the conjugate of penetratin to itself at the concentrations assayed. A control peptide used was a fusion of the human immunodeficiency virus-1 TAT protein (HIV-TAT) delivery peptide sequence (Hileman et al., FEBS Lett. 415:145-154 (1997) and the first helix of the p75 receptor intracellular domain.

### Example 12

### Cell Death Assessment

Primary hippocampal or cortical neuronal cultures or 293 cells transduced with the different penetratin-peptide conjugates were assayed for viability at 24 and 48 h after transduction by trypan blue exclusion or by conversion of 3-[4,5-Dimethylthiazol-2-yl]-2,5-diphenyltetrazolium bromide; Thiazolyl blue (MTT, Sigma, St. Louis) to formazan by dehydrogenase enzymes (MTT, Sigma, St. Louis) and by the LIVE/DEAD assay (Molecular Probes, Eugene, Oreg.), which distinguishes live cells by the presence of intracellular esterase activity, which results in the conversion of the non-fluorescent cell permeant calcein-AM to the intensely green fluorescent calcein. Calcein is retained within live cells. Ethidium homodimer-1 (EthD-1) enters cells with damaged membranes and becomes intensely fluorescent when bonding to nucleic acids. EthD-1 is excluded by the intact plasma membrane of live cells. Media were removed and replaced by 4 μM EthD-1 and 2 μM calcein in PBS. Images were taken 30 min after treatment. The morphology of nuclei in the cultures was examined by staining with 0.1 μg/ml Hoechst 33342.

### Example 13

### Antibodies, Immunostaining and Image Analysis of Hippocampal Cultures

Hippocampal cultures were fixed in 4% paraformaldehyde in 1× phosphate-buffered saline (PBS) for 20 min at room temperature (RT). Cells were then rinsed in 1×PBS and then washed once in 1×Tris-buffered saline (TBS) followed by blocking in 10% donkey serum (Jackson ImmunoResearch Labs, West Grove, Pa.) with 0.1% Triton X-100 in 1×TBS for 1 h at RT. Cultures were incubated overnight in the presence of rabbit anti-GFAP (Sigma, St. Louis) at 1:800 dilution and mouse anti-NeuN (Chemicon, Temecula, Calif.) at 1:100 at 4° C. Negative controls were incubated in 2 mg/ml rabbit and mouse preimmune IgGs (Sigma, St. Louis). All primary antibodies were diluted in 1×TBS containing 10% donkey serum. Cultures were washed for 90 min in 4 changes of 1×TBS and incubated in the presence of donkey anti-rabbit IgG conjugated to Cy3 and donkey anti-mouse IgG conjugated to FITC (Jackson ImmunoResearch Laboratories, Inc., West Grove, Pa.), at 1:250 and 1:400, respectively, in 1×TBS containing 1% donkey serum for 1 h at RT. Cells were washed for 90 min in 4 changes of 1×TBS and mounted in VectaShield-DAPI mounting medium (Vector Laboratories, Burlingame, Calif.). Images were acquired using Nikon Eclipse-800 microscope and Optronics MagnaFire camera and software, and analyzed using Compix Simple PCI software. The total surface area corresponding to red and green fluorescence in each confocal image was determined by image analysis using Simple PCI software (Compix, Inc., Philadelphia).

These experiments showed that C31 induces death of rat hippocampal neurons in primary culture. In order to determine whether C31 is also toxic in primary neuronal cultures, in which transfection efficiencies are relatively low, protein transduction was used. This approach allows for the introduction of polypeptides into cells with an efficiency close to 100%, and utilizes relatively stress-free conditions (Schwarze et al., Trends Cell Biol. 10:290-295 (2000)). The D. melanogaster Antennapedia homeodomain-derived delivery peptide (penetratin) linked by an N-terminal disulfide bond to the APP-derived C31 peptide or to control peptides was used. Disulfide-linkage was chosen over other types of covalent bond in order to allow the C31 peptide to be released intracellularly, in association with reduction of the S—S bond in the intracellular environment.

Hippocampal neuronal cultures derived from 17-day old rat embryos were transduced with 10 μM DP-C31 peptide or the DP control, and a marked decrease in viability observed in the DP-C31-transduced cultures, but not the control cultures, 24 h after transduction. The cells treated with DP-C31 peptide showed prominent cytoplasmic shrinkage and an almost complete disaggregation of the neuritic network. Fluorescence microscopic examination of the same cultures showed a profound reduction in the number of viable cells (cells capable of calcein retention in their cytoplasm), and a proportional increase in the number of cells with damaged membranes permeable to EthD-1. Essentially identical results were obtained for cortical neuronal cultures. To assess the efficiency of transduction, primary hippocampal cultures were transduced with penetratin conjugated to FITC (DP-FITC) and analyzed by confocal microscopy. The DP-FITC peptide was internalized in >95% of the cells in the culture. Incubation of primary hippocampal neurons in the presence of increasing concentrations of DP-C31, but not DP, reduced the number of viable cells capable of converting MTT into insoluble formazan.

Immunocytochemical examination of hippocampal cultures using antibodies specific for a neuronal marker, neuron-specific nuclear protein (NeuN), and a glial marker, glial fibrillary acidic protein (GFAP), revealed a marked decrease in the number of NeuN-immunoreactive cells present in the cultures that had been treated with 10 μM C-C31. No difference in the number of NeuN-reactive cells was found in cultures treated with vehicle, with a control peptide, or with 10 μM C-C31 in the presence of the broad-spectrum caspase inhibitor BAF.

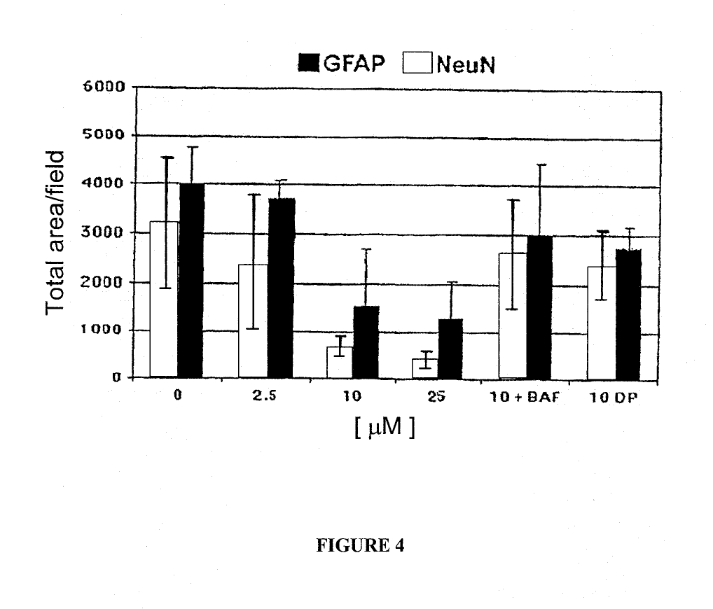

These experiments showed that C31 induces programmed cell death in both neuronal and glial cells. The morphology of the cells that survived transduction with DP was suggestive of glial origin. To investigate whether this was due to a greater sensitivity of neurons than glial cells to DP-APPC31-induced death, 3-day-old rat hippocampal cultures were exposed to increasing concentrations of DP-APPC31 or control DP peptide and fixed 48 h after transduction. The fixed cultures were then immunostained with antibodies specific for GFAP (red) and NeuN (green). A quantitative assessment of the total area of red and green fluorescence present in low-magnification confocal images of representative fields obtained from three independent experiments was performed using a digital image analysis system (SimplePCl, Compix, Inc, Philadelphia). Both the neuronal and the glial populations were significantly reduced in cultures treated with 10 μM DP-APPC3I when compared to untreated or control peptide-treated cultures (see FIG. 4). Transduction with higher concentrations of DP-APPC31 (25 μM) were required to reduce the viability of the neuronal population further, while no further toxicity was observed for glial cells at the concentrations assayed. Incubation in the presence of the broad caspase inhibitor, Boc-aspartyl-fluoromethylketone (BAF), delayed the toxicity resulting from transduction with DP-APPC31, confirming that C31-induced neuronal death in primary cultures is caspase mediated.

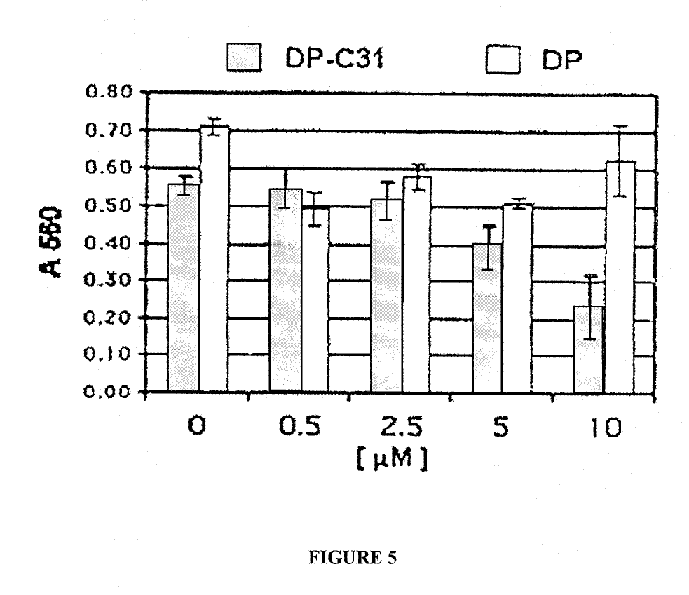

To resolve the discrepancy in the LC₅₀ values obtained by the MTT assay for cell viability (see FIG. 5, 5 μM) and by quantitative image analysis (see FIG. 3, 3.75 μM), the extent of cell death induced by transduction of DP-APPC31 in neuronal cultures was further examined by the trypan blue exclusion method. The LC₅₀ value obtained by trypan blue exclusion for neuronal cultures transduced with DP-APPC31 was 3.75 μM, in agreement with the value obtained by quantitative image analysis (see FIG. 6a). Given that 3-day old cultures of primary neurons were used in all experiments, it is conceivable that the higher LC₅₀ value obtained using the MTT assay was due to variability in the proportion of glial cells present in different batches of primary neurons at the time of plating.

Finally, the number of apoptotic nuclei in cultures transduced with different concentrations of DP-APPC31 was quantitated by Hoechst 33342 staining. An increase in the percentage of condensed, fragmented nuclei present in neuronal cultures was observed when increasingly high concentrations of DP-APPC31 were used for transduction (see FIG. 6b). This observation, together with the finding that APPC31 toxicity was delayed by caspase inhibitors, is consistent with the conclusion that the cellular death induced by the C31 peptide was apoptotic in nature.

Experiments further indicated that exposure to Aβincreases the sensitivity of neurons to C31. It has been shown that exposure of cultured human neuronal and non-neuronal cells to amyloidogenic Aβ peptide induces the activation of apoptotic cell death pathways (Cotman, supra; Cotman and Anderson, supra; La Ferla et al., supra; Nakagawa et al., supra). The concentrations of Aβ used in most of these experiments, however, are likely to be greater than those that may be found in the vicinity of axonal terminals, particularly at early stages in the pathogenesis of AD. It was found that Aβ toxicity in cell lines is augmented by C31. The effect of exposing hippocampal neurons to both Aβ and C31 was therefore assessed. No measurable toxicity was found in hippocampal cultures exposed to concentrations of Aβ alone up to 25 μM. Treatment with 50 μM Aβ, however, was sufficient to decrease the number of viable cells in the culture substantially with a concomitant increase in the number of inviable cells showing permeability for EthD-1.

In the next set of experiments, hippocampal cultures were preincubated in the presence of sublethal concentrations (5 μM) of fibrilar Aβ and these cultures transduced with different peptides 24 h later. Transduction was performed in the absence of Aβ. Preincubation of the primary hippocampal cultures in the presence of 5 μM Aβ for 24 h exacerbated the sensitivity of hippocampal neuronal cultures to C-C31 induced death (LC₅₀<500 nM). The presence of Aβitself could not account for the toxicity observed, since incubation of cells in 5 μM Aβ alone did not increase the number of dead cells present in the culture above background. There was toxicity observed when sublethal concentrations of Aβ and control peptide were added; however, the toxicity observed in cultures treated with Aβ and transduced with C-C31 was likely not due to additive toxic effect (P<0.01 by two-way ANOVA) [[P=0.0059]]. Examination by Hoechst 33342 staining revealed a sharp increase in the percentage of apoptotic nuclei in cultures that had been incubated in the presence of Aβ and then exposed to C-C31 (72%), but not in cultures that had been mock-treated or treated with Aβ (approximately 6% and 9%, respectively).

Aβ exacerbates the sensitivity of hippocampal cultures to C31 toxicity. Primary hippocampal cultures were left untreated or exposed to varying concentrations of Aβ. Thirty-six hours later, the cultures were evaluated by the LIVE/DEAD assay. Primary hippocampal cultures were also tested by preincubation for 24 hours in the presence of a sublethal concentration of Aβ and exposed to varying concentrations of C-C31 or C-C control peptide for an additional 24 hours. Cultures were then assayed by the trypan blue exclusion method. Cells were stained with Hoechst 33342 and scored for nuclear condensation.

### Example 14

### Generation of an APP-Neo Antibody

An antibody that recognizes specifically the epitope generated by cleavage of APP at D664 by caspases was generated at ResGen (Invitrogen Corp., Alabama). Briefly, rabbits were immunized with the peptide ₆₅₇CIHHGVVEVD₆₆4, (SEQ ID NO:6) which includes the nine amino acids immediately preceding the caspase cleavage site at position 664 in APP₆₉₅, coupled to KLH. Antisera from three bleeds over a 10-week period were pooled and affinity purified in three successive steps. (1) Peptide antigen was immobilized on an activated support. Antisera was passed through the column and then washed. After washing, the bound antibodies were eluted by a pH gradient. (2) The eluate from (1) was depleted of immunoglobulins that recognize the intact APP molecule by adsorption to a bridging peptide that encompasses the caspase cleavage site (TSIHHGVVEVDAAVTPEE; SEQ ID NO:7). (3) The flowthrough from (2) was affinity purified on the immobilized immunogenic peptide. After washing, specific antibodies were eluted by a pH gradient, collected and stored in borate buffer. The ELISA titer for this preparation was <1:142,000 (<5 ng/ml) against the immunizing peptide (corresponding to the “novel” C-terminus of APP, an epitope that is generated only after caspase cleavage) versus >1:70 (>10 mg/ml) against the bridging peptide that corresponds to the intact APP sequence across the caspase cleavage site at D664.

### Example 15

### Human Tissue Immunohistochemistry

Human hippocampi obtained from AD or age-matched control patients (Harvard Brain Tissue Resource Center, Belmont, Mass.) fixed with 4% paraformaldehyde were embedded in paraffin. Seven μm microtome sections were deparaffinized in xylene, rehydrated in 100, 95, 80 and 70% ethanol, and washed in 1×TBS for 15 mm at room temperature. A 3% H₂O₂ solution in methanol was used to neutralize endogenous peroxidase-like activity. Microwave antigen retrieval was performed in 10 mM citrate buffer (pH 6.0) for 5 min at 440 watts. Slides were allowed to cool to room temperature and were washed in 1×TBS for 15 min. Samples were blocked in 10% normal horse serum in 1×TBS for 1 hour at room temperature. Primary rabbit IgG to APP-Neo was applied at a dilution of 1:10,000 in 1% BSA in 1×TBS; sections were incubated overnight at 4° C. Rabbit preimmune IgG (Sigma, St. Louis) diluted to 1 μg/mL in the 1% BSA in 1×TBS was used as a negative control. Sections were washed for 30 min in 3 changes of 1×TBS; biotinylated horse anti-rabbit IgG (Vector Laboratories, Burlingame, Calif.) was applied at a dilution of 1:250 for 1 hour at room temperature. Peroxidase-based ABC Elite kit (Vector Laboratories, Burlingame, Calif.) was used according to the manufacturer's instructions followed by a 30 min wash in 3 changes of 1×TBS. A liquid DAB kit (Vector Laboratories, Burlingame, Calif.) was used for the detection; color development was monitored under the microscope. Sections were washed in 1×TBS, briefly counterstained in aqueous hematoxylin, dehydrated, cleared, and mounted in Permount (Fisher Scientific, Pittsburgh, Pa.). Images were acquired using Nikon Eclipse-800 microscope and the Optronics MagnaFire camera and software. Low magnification images were acquired using Nikon SMZ-U dissecting microscope and the CoolSnap camera and software.

The APP-neo antibody of the present invention was used to show caspase-cleaved APP in the brains of patients with AD and control, non-AD patients. To document the generation of C31 peptides in cultured cells and tissues, an antibody was generated that is capable of recognizing exclusively the novel epitope that arises by caspase cleavage of APP at its C terminus (APP-Neo), as described in Example 14. Hippocampi obtained from AD or age-matched control subjects were examined by immunohistochemistry using the APP-Neo antibody. Hippocampal sections from AD brains showed that APP-Neo immunoreactivity, indicative of cleavage of APP at its C-terminus, is intense anteriorly in the polymorphic layer, reduced in the stratum granulosum, decreased in CA4-CA2 and absent from the stratum moleculare. APP-Neo staining was less intense at more posterior levels, but could be detected as dense deposits and in efferent fibers near CA3. Staining was abolished if the primary antibody was preadsorbed with the immunizing peptide, but not if it was preadsorbed with a peptide that encompasses the immunizing peptide sequences and the first 5 N-terminal amino acids of the C31 peptide, past the caspase cleavage site (bridge peptide). Specific APP-Neo immunostaining occurred in the hippocampus of a 90 y.o. without AD as well (i.e., control brain), but to a lesser degree, staining was low to moderate in cells and fibers of the polymorphic layer and stratum granulosum, declining in CA4-CA2 and absent from the stratum molecular. In contrast to the AD brains, no APP-Neo staining could be detected at more posterior levels in the hippocampus. Staining was abolished by preadsorption with the immunogenic peptide, but not by preadsorption with bridge peptide.

### Example 16

### Analysis of APP-Like Proteins

APLP1 and APLP2 are cleaved by caspases. Three members of the APP family of proteins exist: APP, APLP1 and APLP2. Even though the overall similarity of the APP family C-termini is not high, the caspase cleavage site that is required for the generation of APPC31 is completely conserved in all three members. If the DEVD sequences in APLP1 and APLP2 can function as caspase cleavage sites, both proteins could potentially generate C-terminal peptides. It should be noted, however, that the P1′ position in APP is Ala (VEVDA; SEQ ID NO:8), whereas the P1′ position in APLP1 is Pro (VEVDP; SEQ ID NO:9) as it is in APLP2. Caspases tend to prefer less bulky residues such as Gly, Ala, or Ser, in the P1′ position, rather than more bulky residues such as Pro. Therefore, at least in theory, the VEVD site in APP should be more readily cleaved by caspases than the sites in APLP1 and APLP2. To determine whether APLP1 and APLP2 can be cleaved by caspases, a panel of recombinant caspases were assayed for their abilities to cleave ³⁵S-labelled, in vitro transcribed/translated APP, APLP1 and APLP2. The results show that APP can be cleaved by caspases-3 and -6, but not by caspase-8. APLP1, on the other hand, was cleaved in vitro only by caspase-3, not by caspase-6 or -8. Like APLP1, APLP2 may be cleaved in vitro by caspase-3 only, but with very low efficiency, if at all. The ³⁵S-Met-labelled C31 peptide product of the cleavage of APP by caspases-3 and -6 was detected as a ˜4 kDa band. However, the homologous peptide generated by caspase-3 cleavage of APLP1 was not detectable, likely due to the fact that only one methionine (of a total of two in APPC31) is conserved in APLP1C31.

To determine whether cleavage of APLP1 and APLP2 can occur in cultured cells, CMV-driven constructs expressing N-terminally FLAG-tagged APLP1 and APLP2 or a full-length APP construct were transfected in 293 cells and activated the caspase cascade by treatment with staurosporine. Both APP and APLP1 were cleaved in staurosporine-treated 293 cells and in both cases, cleavage was prevented by incubation of the cells in the presence of BAF. Both the full-length and the truncated forms of APP were detected. Full-length APLP1 appeared to be completely degraded in 293 cells treated with staurosporine, but not when BAF was present. No cleavage products of FLAG-APLP1 could be detected in these cultures. Both in vitro and in transfected 293 cells, caspases could cleave APP and APLP1 at more than one site. No evidence was found for the cleavage of FLAG-APLP2 in transfected 293 cells.

To determine whether APLP1 is effectively cleaved at position 664, the selective reactivity of the APP-Neo antibody was used. Given that the 5 amino acids that constitute the novel C-termini in cleaved APLP1 and APLP2 are relatively conserved, epitopes could be generated that might be recognized selectively by APP-Neo after caspase cleavage. To determine whether APP-Neo immunoreactive epitopes are generated by caspases in APP, APLP1 and APLP2, unlabelled, in vitro transcribed/translated full length APP (APP₆₉₅), APPD₆₆₄A (a mutant of APP in which the D residue at position 664 has been replaced by A), and full-length APLP1 and APLP2 were incubated in the presence of recombinant caspases. The products of the reactions were separated on polyacrylamide gels and immunoblotted with APP-Neo antibody. Control immunoblots were performed using lysates from 293 cells transfected with full length APP₆₉₅, with an APP construct lacking the APP C-terminal 31 amino acids (APPdeltaC31), or with APP₆₉₅ and treated with 10 μM staurosporine, in the presence or absence of 50 μM BAF. APP-Neo immunoreactive bands were detected only in lysates from 293 cells expressing APPdeltaC31 and in lysates from cells expressing APP₆₉₅ and treated with staurosporine in the absence of BAF. Also, an APP-Neo-immunoreactive epitope was detected in immunoblots of in vitro transcribed/translated full length APP that had been incubated in the presence of recombinant caspase-3, but not caspase-7 or -8. Likewise, in vitro transcribed/translated APLP1 and APLP2 yielded APP-Neo immunoreactive cleavage products only when incubated in the presence of recombinant caspase-3. Providing a control for the specificity of the reaction, a mutant form of APP that cannot be cleaved by caspases, APPD₆₆₄A, did not yield detectable APP-Neo immunoreactive products after incubation with recombinant caspase-3, -7 or -8.

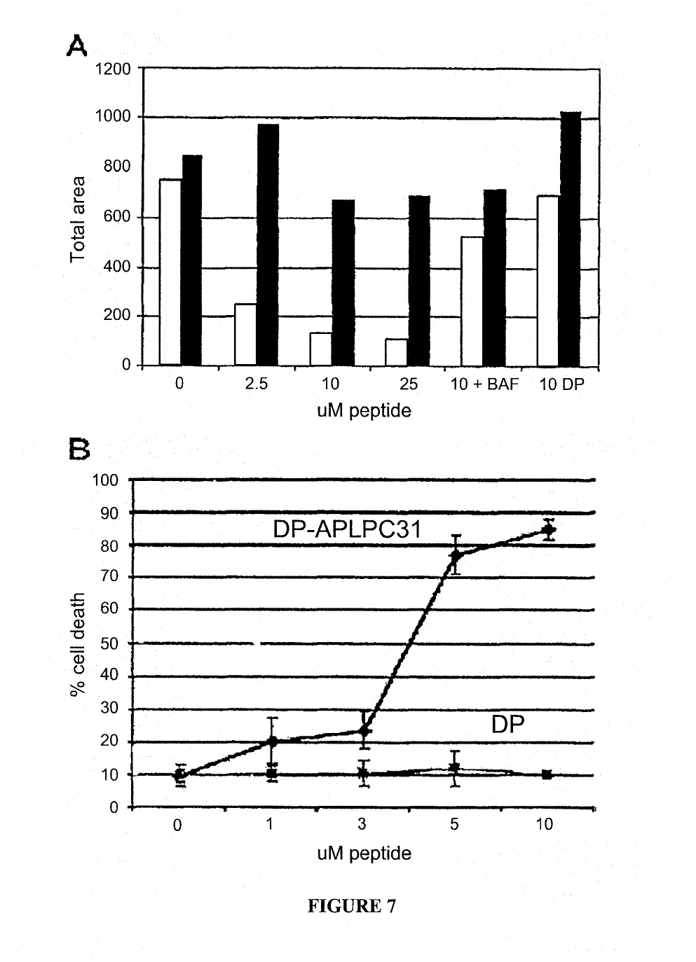

APLP1C31 induces death in primary hippocampal cultures. The results presented suggest that APLP1 can be cleaved by caspase-3 at the aspartic acid residue at position 620. If this event occurs in vivo, APLP1 would have the potential to generate a pro-apoptotic C-terminal peptide homologous to APPC31. To determine whether the peptide generated by caspase cleavage of APLP1 is toxic, a fusion of APLP1C31 to the Antennapedia delivery peptide (DP-APLP1C31) was generated and assayed in protein transduction experiments. Three-day-old rat hippocampal cultures were exposed to increasing concentrations of DP-APLP1C31 or control peptide, fixed 36 h after transduction and immunostained with antibodies specific for GFAP and NeuN. A quantitative assessment of the relative areas of red (GFAP) and green (NeuN) fluorescence present in low-magnification confocal images was performed using a digital image analysis system (SimplePCl, Compix, Inc, Philadelphia). The NeuN-immunoreactive population was markedly reduced in cultures treated with 10 μM DP-APPC31 (see FIG. 7a). At higher concentrations of DP-APPC31 (25 μM), the viability of the neuronal population was reduced further. A modest decline in the viability of glial cells was observed, which may have been due to a relatively higher sensitivity of neurons to APLPC31 toxicity. Incubation in the presence of the broad caspase inhibitor, Boc-aspartyl-fluoromethylketone (BAF), delayed DP-APLP1C31 toxicity, consistent with the suggestion that cell death induced by APLP1 C31 depends on caspase activity.

The extent of cell death induced by transduction of DP-APPC31 in neuronal cultures was further examined by the trypan blue exclusion method. As shown in FIG. 7b, a dose-dependent reduction in the viability of the cultures was observed at increasing concentrations of transduced DP-APLP1C31 but not of control DP peptide. The LC₅₀ value obtained for neuronal cultures transduced with DP-APPC31 was 4 μM, while the value obtained by image analysis was approximately 5 μM.

### Example 17

### Identification of Non-Peptide Small Molecule Compounds Competing for the Specific Binding of the PTB Domain of X11 and the C-Terminal Domain of APP

Since the C-terminal part of APP plays a critical role in both APP internalization and in the induction of cell death, and it has been shown herein that C31 (peptide sequence: AAVTPEERHLSKMQQNGYENPTYKFFEQMQN; SEQ ID NO:1) is capable of inducing cell death in cells expressing APP (Lu et al., supra), a rational approach has been employed for the identification of peptide and non-peptide small molecules that will compete for the specific binding of Fe65/APP and X11/APP (employing, for example, Catalyst, software from Molecular Simulations Inc.). For this purpose, the available crystallographic structure of X11/APP complex are used and the Fe65/APP interaction modeled based on the X11/APP complex. Applying this approach, 145 potential pharmacophores have been identified from 5 databases containing more than 600,000 compounds. Four of these have a statistically significant docking score and can be grouped in two chemically distinct groups, i.e., flavonoids and antibiotics. Additional potential compounds contemplated for use in the practice of the present invention include small molecules such as, for example, peptides, peptidomimetics, antisense peptides, antibodies, antagonists, antisense nucleic acids, and the like.

While the invention has been described in detail with reference to certain preferred embodiments thereof, it will be understood that modifications and variations are within the spirit and scope of that which is described and claimed.

All references cited herein are hereby incorporated by reference in their entirety.

1-22. (canceled)

23. A method of identifying an agent that blocks cleavage of APP at Asp 664 (APP 695 numbering) or an APP-like protein (ALP) at sequence VEVDP after aspartic acid, said method comprising (a) contacting a candidate agent to a composition comprising APP or ALP and (b) determining whether the candidate agent blocks cleavage of APP or ALP in the composition as indicated by specific binding to APP or ALP or decreased production of C31 peptide compared to a control composition without the candidate agent.

24. The method of claim 23, wherein the agent is a small molecule.

25. The method of claim 24, wherein the small molecule is selected from the group consisting of a peptide, an antisense peptide, a peptidomimetic, or an antibody.

26. The method of claim 23, wherein candidate agent decreases production of C31 peptide compared to a control composition without the candidate agent.

27. A method of identifying an agent that blocks cleavage of APP at Asp 664 (APP 695 numbering) or an ALP at sequence VEVDP after aspartic acid, said method comprising (a) contacting a candidate agent with a transgenic cell comprising a transgene encoding APP or ALP and (b) determining that the candidate agent blocks cleavage of APP or ALP in the first transgenic cell as indicated by specific binding to APP or ALP, increased cell viability, decreased apoptosis, or decreased production of C31 peptide compared to a control transgenic cell without the candidate agent.

28. The method of claim 27, wherein the transgenic cell is a neuronal cell.

29. The method of claim 27, wherein the agent is a small molecule.

30. The method of claim 29, wherein the small molecule is selected from the group consisting of a peptide, an antisense peptide, a peptidomimetic, or an antibody.

31. The method of claim 27, wherein the candidate agent increases cell viability compared to a control transgenic cell without the candidate agent.

32. The method of claim 27, wherein the candidate agent decreases apoptosis compared to a control transgenic cell without the candidate agent.

33. The method of claim 27, wherein the candidate agent decreases production of C31 peptide compared to a control transgenic cell without the candidate agent.


SEQUENCE LISTING (11 sequences)

SEQ ID NO: 1 - protein, 31 aa, Artificial Sequence
  Synthetic Peptide
AAVTPEERHLSKMQQNGYENPTYKFFEQMQN

SEQ ID NO: 2 - protein, 30 aa, Artificial Sequence
  Synthetic Peptide
PMLTLEEQQLRELQRHGYENPTYRFLEERP

SEQ ID NO: 3 - protein, 31 aa, Artificial Sequence
  Synthetic Peptide
PMLTPEERHLNKMQNHGYENPTYKYLEQMQI

SEQ ID NO: 4 - protein, 16 aa, Drosophila melanogaster
RQIKIWFQNRRMKWKK

SEQ ID NO: 5 - protein, 4 aa, Artificial Sequence
  Synthetic Peptide
NPTY

SEQ ID NO: 6 - protein, 10 aa, Artificial Sequence
  Synthetic Peptide
CIHHGVVEVD

SEQ ID NO: 7 - protein, 18 aa, Artificial Sequence
  Synthetic Peptide
TSIHHGVVEVDAAVTPEE

SEQ ID NO: 8 - protein, 5 aa, Artificial Sequence
  Synthetic Peptide
VEVDA

SEQ ID NO: 9 - protein, 5 aa, Artificial Sequence
  Synthetic Peptide
VEVDP

SEQ ID NO: 10 - protein, 4 aa, Artificial Sequence
  Synthetic Peptide
DEVD

SEQ ID NO: 11 - protein, 4 aa, Artificial Sequence
  Synthetic Peptide
VEVD